(19) United States
(12) Patent Application Publication
(10) Pub. No.: US 2010/0042034 A1
(43) Pub. Date: Feb. 18, 2010

(54) WOUND CARE PRODUCT, COMPRISING TEXTILE RIBBONS WITH FIBRES HAVING GEL-FORMING PROPERTIES, AND FIBRES NOT HAVING GEL-FORMING PROPERTIES

(76) Inventor: Birgit Riesinger, Ostbevern (DE)

Correspondence Address: OCCHIUTI ROHLICEK & TSAO, LLP, 10 FAWCETT STREET, CAMBRIDGE, MA, 02138, US

(21) Appl. No.: 12/539,998

(22) Filed: Aug. 12, 2009

(30) Foreign Application Priority Data
Aug. 15, 2008 (DE) 102008037888.7-45

(51) Int. Cl. A61F 13/53; D05B 1/00; D03D 23/00; D04B 39/00

(52) U.S. Cl. 602/44; 602/43; 602/45; 602/46; 112/475.01; 139/1 R; 66/1 R

(57) ABSTRACT

The subject matter of the invention is a wound care article, exhibiting fibers with gel-forming properties containing at least one planar structure, as well as fibers with non-gel-forming properties, characterized in that the fibers are arranged in the form of planar fabric bands.

The invention relates to a wound care article according to the characterizing clause of Claim 1.

Such a wound care article is in particular suitable for the absorption of exudate from chronic wounds, such as occur e.g. in diabetes, leg and foot ulcers and similar diseases.

The term “exudate” describes a wound fluid discharged from blood plasma via the inflammatory processes of the wound edema. Just as the blood is responsible for the transport of nutrients and other messenger substances and with this for the care of different parts of the body, the exudate, in quite similar fashion serves the purpose of the care of the wound bed and of the healing processes proceeding in it. In order to fulfill this multitude of functions, it contains a broad spectrum of components, from which a specific weight results, said weight lying slightly above that of water. In this sense it also differs from transudate, which is discharged from non-inflammatory processes and exhibits a clearly lower specific weight with a low cell and protein content. Along with the provision of nutrients for the fibroblasts and epithelial cells the exudate coordinates the different processes of wound healing chronologically and spatially through its high content in growth factors and cytokines. These are formed above all by thrombocytes, keratinocytes, macrophages and fibroblasts. They influence the motility, migration and proliferation of the various cells participating in the wound healing. Thus the immigration of cells into the base of the wound is promoted as well as the provision of the newly formed granulation tissue by the angiogenesis. The exudate also supports the wound cleansing procedure. It contains various serine, cysteine and aspartate proteases as well as matrix metalloproteases, which strictly regulated in their activity, break down irreversibly damaged tissue and hence prepare the wound bed for the subsequent phases of healing.

Components of the physiological exudate are in particular salts, glucose, cytokines and growth factors, plasma proteins, proteases (in particular matrix metalloproteases), granulocytes and macrophages.

If there is not a clear progression of the wound healing process within a few weeks corresponding to the various phases of wound healing, one speaks of a chronic wound. In the process however one considers exudative phases lasting longer than three days as complications and speaks of a pathological exudation, which can contribute to a chronification of the wound. The underlying causes are usually complex and can definitely also be of a systemic nature. However, on the basis of the previously described significance of the exudate for wound healing it is no surprise that complications of wound healing can be reflected in a significantly altered composition and effect of the exudate.

Among other things through a concentration shift of the individual components of the exudate the ordinarily curative exudate loses its positive effect in the case of chronic wounds. In particular the content in inflammatory cytokines and proteases is significantly increased in pathological exudate. The content in growth factors on the other hand is reduced. A particularly serious difference arises with regard to the activity of the matrix metalloproteases previously addressed. Along with the preparation of the wound beds these also participate in the subsequent conversion of the granulations tissue to scar tissue. These enzymes are ordinarily formed as inactive pre-enzymes and are regulated in their activation by corresponding inhibitors (tissue inhibitors of metalloproteases, TIMPs) which simultaneously themselves have a positive effect on the cell growth. In the chronic exudate the activity of the proteases appears increased on the basis of failures in this regulatory system, a fact which possibly contributes to an active wound regression. The pathological exudate, with regard to the content of its components has fallen out of the balance promoting the wound progression. As a result of this various complications arise which contribute to the further deterioration and chronification of the wound.

In recent times gel-forming polymers have been used increasingly in wound management. Said gel-forming polymers on the one hand exhibit a high binding capacity for the named exudates, but simultaneously form a non-adherent and pleasantly cooling gel surface. Carboxymethycellulose (CMC) is used especially frequently.

Carboxymethycelluloses are derivatives of cellulose, in which case a part of the hydroxyl groups of the cellulose are linked as ether with a carboxymethyl group. In the production the cellulose is for example transformed in to more reactive alkali cellulose and subsequently converted with chloroacetic acid into carboxymethyl cellulose. The cellulose structure remains preserved. In its acid form CMC is insoluble in water. Under alkaline conditions on the other hand they are relatively well soluble in water.

CMC can absorb up to circa four times its own volume in liquid before colloid formation takes place. The liquid absorption occurs, unlike as in the case of alginates, in which Ion exchange processes take place, rapidly, wherein the liquid and components dissolved within become firmly integrated, until the fiber is supersaturated.

Carboxymethyl cellulose is present in particular in the form of sodium carboxymethyl cellulose. In hygiene and wound products the fibers are transformed into a planar matrix. Through the absorption of liquid from the wound exudate the fibers are little by little transformed to a gel cushion which holds the liquid and does not release it. In the process the fibers are structured in such a way that the wound exudate is only absorbed in vertical direction. This means that as long as the capacity suffices, the exudate does not flow beyond wound edge. In this way wound edge maceration can be effectively prevented.

The use of wound care articles exhibiting CMC is in addition suitable in particular for the
Treatment of severely exudative wounds
Support of the wound healing processes by the maintenance of a humid environment
Hemostatic properties

### STATE OF THE ART

A wound dressing is known from EP12166319 in which case a gel-forming yarn, for example made of carboxymethyl cellulose (CMC), is interwoven with a non gel-forming yarn, for example nylon, wherein the latter serves as a stabilizing yarn in order to guarantee the structural integrity of the wound dressing even under the influence of moisture.

From DE 69530180 a non-adherent bandage in web form is known, which exhibits from 50 percent by weight to 95 percent by weight textile fibers mixed with from 5 percent by weight to 50 percent by weight gel-forming fibers. The textile fibers named in the publication, for example cellulose fibers exhibit a relatively weak absorptive capacity, while the fibrous polymers have an increased absorptive capacity, which should range up to 50-fold of the dead weight. Disadvantageous in the case of the known bandage is the fact that both the textile as well as also the gel-forming fibers exhibit insufficient absorption capacity for specified applications, for example for very severely exudative wounds. Therefore this bandage exhibits only a low absorption capacity for wound exudates. Moreover the absorption behavior is difficult to dose.

Methods for the chemical modification of cellulose to such an extent that it exhibits gel-forming properties are known from the state of the art.

Thus in general a method for the carboxyalkylation of cellulose is known which in particular leads to the production of carboxymethyl cellulose. In the process cellulose (unbranched polymer made of 1-4-β-glycosidic linked glucose molecules with a chain length between one hundred and ten thousand monomers) is converted by alkali-catalyzed reaction with a chloroalkanoic acid (for example chloroacetic acid). The polar carboxyl groups make the cellulose soluble and are responsible for the gel formation. The functional properties of CMC depend on the degree of substitution of the cellulose, i.e. on the number of carboxymethyl groups per glucose subunit.

The disadvantage of this method is that—if the maintenance of precisely controlled process conditions is sought, as is required for the precise setting of a homogeneous degree of substitution and with this of the gel-forming properties—this method cannot be applied for planar cellulose structures, but rather only for dispersed cellulose fibers or for very narrow cellulose bands. This also holds true for the wound dressing in accordance with the above named documents constituting the state of the art.

For example, with this method it is possible to controlledly carboxymethylate tubular cellulose fabric or knitted fabrics with a tube diameter of a maximum of 5 cm. A product manufactured thus is commercially available under the brand name “Rapid Rhino Sinu Knit”. If one wanted to produce larger structures with this method, the process-technical expenditure which is required in order to continue to guarantee a homogeneous degree of substitution would increase disproportionately. For this reason it is not economically practical to treat larger structures with the named method.

Planar structures, as for example required for use as large area wound care article (e.g. 10×10 cm), cannot be carboxymethylated with the known methods from the state of the art.

### OBJECT OF THE PRESENT INVENTION

The object of the present invention is to provide a wound dressing which does not exhibit the named disadvantages. This problem is solved with the features of the independent claims.

According to this a wound care article, exhibiting at least one planar structure containing fibers with gel-forming properties, as well as fibers with non-gel-forming properties, is provided which is characterized in that the fibers are arranged in the form of planar fabric bands.

The term “fibers”, as it is used here refers
a) both to textile individual fibers (so-called monofilaments), as well as also
b) to polyfilaments produced from these monofilaments, in particular spun or twisted yarns.

In the process the aforementioned monofilaments are preferably not present in spun or twisted form—thus for example as yarn—but rather are processed directly as monofilaments to the aforementioned fabric bands.

The term “planar fabric band”, as used here, refers to a planar Band, which consists of fibers in the above terms. In particular provision can be made here that a Fabric band

consists
a) only of fibers with non-gel-forming properties,
b) only of fibers with gel-forming properties, or
c) both of fibers with non-gel-forming properties as well as also of fibers with gel-forming properties

The aforementioned fabric bands preferably exhibit widths in the range of and including 1 mm and including 8 cm auf, especially preferably between and including 5 mm and including 3 cm.

Preferably provision is made that the aforementioned wound care article exhibits several planar fabric bands which are which are interwoven, interlaced, stitch-bonded or interlinked. Examples of these embodiments are shown in the figures.

In this way a planar arrangement with randomly dimensioned square measures can be produced which exhibits a handy, rough structure which the patient senses as being pleasant. In addition there is the fact that e.g. by corresponding spatial arrangement of the bands (for example in the formation of a plait, see figures) the aforementioned arrangement can exhibit a three-dimensional structure, which the patient likewise senses as being pleasant.

Here in particular provision can be made that in the case of the mentioned processing (interweaving, interlacing, stitch-bonding, interlinking) passages between the bands are left. This is for example important if the named planar arrangement is used as a sleeve around an absorption body which contains highly absorbent substances such as e.g. super-absorbent polymers. The passages prevent the arrangement which is formed in the case of contact with liquid partially forms a gel, from being impassable for liquids. Instead of this the passages ensures that even in moist state liquid can pass through the arrangement and reach the absorption body.

As an alternative provision can be made that in the aforementioned fabric bands at least one elastic Band is arranged in longitudinal direction—for example a heavy/thick Spandex fiber Band—, in such a way that the bands take on a pleated and/or ruffled structure.

In principle provision can be made that
a) the fabric bands without exception consist both of fibers with non-gel-forming properties as well as also of fibers with gel-forming properties, or that
b) the fabric bands only of fibers with non-gel-forming properties are interwoven with Fabric bands only of fibers with gel-forming properties.

Preferably in the process provision is made that the fibers with gel-forming properties are based on chemically modified cellulose.

In the case of cellulose it is a matter of an unbranched polymer made of 1-4-β-glycosidic linked glucose molecules with a chain length between one hundred and ten thousand monomers. Formula I shows as an example a section from a cellulose molecule.

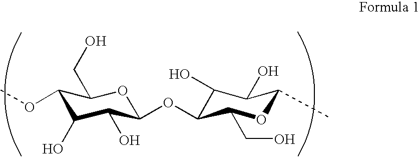

The known viscose (cellulose xanthate, “Rayon”) is likewise included in the term “cellulose” as defined by the present invention. Viscose is produced by means of the treatment of cellulose with 18-22-% sodium hydroxide solution, wherein first alkali cellulose forms, the sodium salt of the cellulose. The alkali cellulose is pressed and reduced to fibers. In the next work step the alkali cellulose is transformed with carbon disulfide, wherein cellulose xanthate forms. In this connection a substitution takes place at one or more OH-groups of the glucose monomers. In order to obtain viscose for the production of fibers, in the process on the average there must be one carbo disulfide substituent for two glucose monomers.

Subsequently a so-called “spinning solution” is produced. For this purpose the obtained cellulose xanthate is dissolved in 7% sodium hydroxide solution, degassed and pressed through fine nozzles into a solution of sulfuric acid and sulfates. In this solution the carbon disulfide molecules, which are bound to the cellulose, are in large part separated again, and the viscose fibers are formed. Formula 2 shows as an example a section of a viscose molecule.

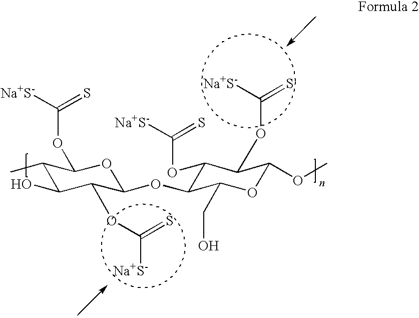

The arrows in Formula 2 refer to the xanthate substituents; the frequency of said xanthate substituents being exaggerated, since in the case of viscose as already said on the average there is one carbon disulfide substituent for two glucose monomers.

Additional substances that fall into the category of “cellulose” as defined by the present invention are
Lyocell (a fiber material artificially manufactured out of cellulose which is produced by the dissolving of cellulose in a solvent, as a rule N-methylmorpholine-N-oxide, and subsequent spinning (“solvent spinning”), and
Polynosic (fibers manufactured from regenerated cellulose in accordance with a modified viscose spinning process, which in comparison to viscose exhibit a higher wet strength, higher alkali resistance and a lower swelling capacity).

The fibers with non-gel-forming properties preferably exhibit chemically modified cellulose.

Preferably in the case of the chemically modified cellulose it is a matter of at least partially substituted cellulose, preferably cellulose ether, such as e.g. alkylated cellulose (e.g. cellulose methyl ether, cellulose ethyl ether, propyl cellulose), hydroxyalkylated cellulose (e.g. hydroxymethyl-cellulose, hydroxyethyl-cellulose, hydroxymethyl-cellulose, hydroxypropyl-methyl-cellulose) or carboxyalkylated cellulose (e.g. carboxymethyl cellulose, carboxycellulose ethyl ether, carboxypropyl cellulose).

Preferably provision is made that the modified cellulose exhibits a degree of substitution between and including 0.05 and including 3 alkyl, hydroxyalkyl or carboxyalkyl groups per glucose unit. In the process a degree of substitution of 3 denotes three substituents per glucose unit, while a degree of substitution of 0.05 denotes one substituent per 2000 glucose units.

Aforementioned chemically modified celluloses upon contact with water are converted successively into a gel-like state. In the process the gel-forming property depends on the degree of substitution of the cellulose, i.e. the higher the degree of substitution, the earlier the gel formation occurs. With the help of the degree of substitution, however; the occurrence of the loss of the structural integrity of the chemically modified cellulose can also be controlled. By this it is meant that the gel formation of the chemically modified cellulose in the most extreme case results in the chemically modified cellulose being transformed into a completely amorphous gel no longer to be described as a structurally integral unit, which under circumstances can no longer be removed from the wound as a unit, but rather e.g. must be scraped off with the help of a spatula.

The loss of the structurally integrity occurs relatively early in the case of a high substituted cellulose, while it occurs relatively late or not at all in the case of a relatively low substituted cellulose.

The chemically modified cellulose can likewise be oxidized cellulose. Such oxidized cellulose is e.g. known from EP1215217. It exhibits similar properties to carboxyalkylated cellulose, however can be manufactured more cost-effectively and, what is more, from recycled material.

In general it holds true that the above named methods for chemical modification of cellulose are also applicable to viscose. As already explained above, viscose exhibits on the average one carbon disulfide substituent for every two glucose monomers. Thus there are sufficient free OH-groups present which can be substituted in the above named manner, preferably carboxyalkylated (carboxymethylated) or oxidized.

In the case of the chemically modified cellulose it is preferably a matter of Carboxyalkyl cellulose or carboxyalkyl viscose. Formula 3 shows as an example a section from a carboxymethyl cellulose molecule.

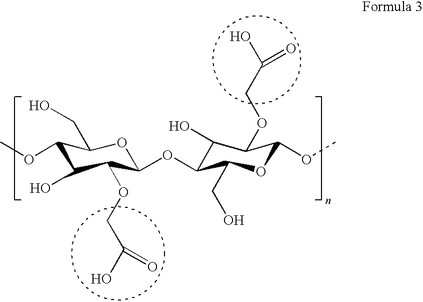

The arrows refer to the substituents, which are carboxymethyl groups (—CH₂—COOH). The degree of substitution here amounts to 1 per glucose unit.

Here too it is pointed out that for the purpose of the invention other OH groups can be substituted by xanthate substituents. In this case the aforementioned molecule is carboxymethyl viscose, likewise conceivable are similarly substituted fibers based on Lyocell or Polynosic.

As already mentioned, in accordance with the invention the degree of substitution can lie within the range of and including 0.05 to and including 3. In the case of the substituents, deviating from what has been said earlier it can also be a matter of other alkyl, hydroxyalkyl or carboxyalkyl groups; in this case it is then a matter of the corresponding die entsprechenden analog modified celluloses.

Preferably provision is further made that the fibers with non-gel-forming properties maintain their structural integrity in spite of the influence of moisture.

In particular these fibers do not form a gel when they come into contact with moisture. Preferably these fibers exhibit at least one at least one fiber type selected from the group containing cellulose, viscose, linen, wool, and/or synthetic fibers, such as e.g. polyamide fibers (“Nylon”), polyester fibers, polyacrylic fibers, polypropylene fibers, Spandex fibers (“Lycra”) and the like.

Here again a clear distinction must be made between non-chemically modified cellulose (or viscose), which does not exhibit any gel-forming properties, and chemically modified cellulose (or viscose) in accordance with the above description (or viscose), which exhibits gel-forming properties.

The aforementioned non-gel-forming fibers thus have an essentially supporting function here which prevents the wound care article from completely transforming to a gel upon contact with liquid (for example wound exudate) which can no longer be removed from the wound as a whole. Said non-gel-forming fibers thus ensure the maintenance of the structural integrity of the wound care article according to the invention. This is in particular important since in this way also components of the wound care article converted to gel form can be removed with said wound care article from the wound.

Important for the latter in particular is the fact that—as provided in accordance with the invention—gel-forming fibers and non-gel-forming fibers are arranged in immediate spatial relationship to one another.

Preferably provision is further made that the amount of fibers with gel-forming properties at the aforementioned planar structure of the wound care article lies within the range between and including 50 percent by weight and 100 percent by weight.

Especially preferably provision is made that the aforementioned planar structure exhibiting planar fabric bands
a) forms a sleeve around an absorption body,
b) forms the shape of a dot, and/or
c) forms the shape of a wound cloth, a wound dressing, a wound compress, a wound cushion, a bandage or a stocking.

Furthermore provision is preferably made that the wound care article additionally exhibits super-absorbent polymers.

Super-absorbent polymers (SAP) are plastics which are able to absorb a multiple of their dead weights—up to 1000 times—in liquids. From a chemical standpoint an SAP is a copolymer made of acrylic acid (propenoic acid, C₃H₄O₂) and sodium acrylate (sodium salt of the acrylic acid, NaC₃H₃O₂), wherein the ratio of the two monomers to each other can vary. Additionally a so-called core cross linker (CXL) is added to the monomer solution which bonds the formed long-chain polymer molecules to each other in places by chemical bridges (“cross-links” them). Through these bridges the polymer becomes water insoluble. In the case of the penetration of water or aqueous salt solutions into the polymer particles, they swell up and tauten on the molecular plane of this network, so that the water can no longer escape without help.

The superabsorber particles can be present in the form of powder or granulate of a particle size ranging between 100 and about 1000 μm.

Preferably the superabsorbent polymers are polymers in the form of fiber, yarn, cotton, fleece or fabric. In another preferable embodiment the superabsorbent polymers are polymers in the form of powder or granulate.

Furthermore provision is preferably made that the wound exudate absorption body exhibits at least one material which is selected from the group containing
a) a fiber mat, in particular made of an airlaid, with incorporated superabsorbent polymers,
b) a fleece, a non-woven, an airlaid, a mat, a knitted fabric or a fabric exhibiting fibers or yarns made of superabsorbent polymers,
c) an absorbent cellulose material
d) a loose filling exhibiting super-absorbent polymers, and/or
e) a mat made of flexible foam.

Significant advantages of this configuration are
that the sleeve itself can absorb considerable quantities of wound exudates, so that the overall absorption capacity of the wound care article increases;
the sleeve exhibits a slight adhesion to the wound due to the amount of gel-forming fibers;
the sleeve exhibits seam strength through the through the amount of supporting fibers,
in the use of gel-forming fibers the sleeve exerts a pleasantly cooling and remoisturizing effect on the wound;
the supporting fibers ensure the integrity of the sleeve even in the case of the parallel use of gel-forming fibers; and
an absorption gradient develops between the absorption body situated inside and sleeve, wherein the interior absorption body serves as a reservoir and a complete disintegration of the sleeve is absent, because excess liquid is directed inward.

The term “airlaid” refers to a special non-woven fabric made of pulp and polyolefin fibers, in which if applicable super-absorbent polymers are embedded.

It can be constructed in mat form and preferably exhibits a liquid permeable sleeve which e.g. is made of polypropylene.

The superabsorbent polymers can in the process be present in the form of fiber, yarn, cotton, fleece or fabric, in the form of powder or granulate or in the form of foam, packing material, a pressed article or a packing material made of shreds consisting of a cut up airlaid mat.

In a further preferable embodiment provision is made that the wound exudate absorption body exhibits at least one material selected from the group containing a mat, in particular made of airlaid, with incorporated superabsorbent polymers, and/or a loose packing material of superabsorbent polymers. Aforementioned airlaid mat has already been described above and can preferably exhibit an essentially flat material section made of absorption material, which e.g. consists of an absorbent fleece from the named fibers with superabsorbent polymers distributed inside.

This wound exudate absorption body can correspond to the absorbing liner which is contained in a in a wound dressing of the applicant of the present invention, as for example is disclosed in WO03094813, WO2007051599 and WO0152780 and which is marketed under the trade name “sorbion sachet”. The disclosure of the named publications is attached in its entirety to the disclosure of this publication.

The wound exudate absorption body can in another embodiment likewise form a core which consists of if applicable flocculent—cellulose and/or cellulose derivatives, preferably flock pulp or synthetic wool fibers, superabsorbent polymers in granulate form as well as an adhesive, wherein the granulates are adhered to the cellulose or the pulp at several elevations, and the granulates are distributed over more than 50% of the total overall height of at least one section of the core, wherein blended regions of granulate and cellulose and/or cellulose derivatives are present. The percentage by weight of the superabsorbent polymers can in the process preferably range between 10-25 percent by weight. Similar constructions are known from conventional incontinence materials and are known for their cushioning properties in the case of hygienic dressings.

A sleeve can be arranged around the aforementioned core, which is arranged overlapping in regions and which e.g. covers a bonded seam or is part of the same.

Likewise, a section of a hydrophobic and/or water-repellent or waterproof material can be provided within the sleeve, said section acting as drench or laundry protection.

The wound exudate absorption body can in another embodiment likewise contain at least one flat layer of a pulp to which superabsorbent polymers—preferably in granulate form—are adhered. As a result of this in a preferable embodiment a structure of the body arises which exhibits at least three layers, wherein two covering layers enclose one layer exhibiting superabsorbent polymers.

In the process on the flat side there are no blends of pulp and superabsorbent polymers; but rather only fixed adjacent areas of both materials. The several layers provided if applicable can in the process in a preferable embodiment also be compressed with each other by rolling, pressing, calendering or similar methods.

Moreover the body can exhibit repeating patterns or grains, such as e.g. a check pattern, an embossed pattern or the like.

The aforementioned wound exudate absorption body can moreover if applicable exhibit a sleeve made of a permeable material; said sleeve can exhibit different connections or seams in its peripheral regions, said bonds or seams being generated in particular by bonds. For example, provision can be made that the connection region between the two sides of the sleeve on at least one side of the wound exudate absorption body—preferably in longitudinal direction—is formed narrower than on at least one other side so that in the first case, otherwise than in the latter case a protrusion which can be unfolded results.

Aforementioned sleeve can preferably be made of a non-woven made out of polypropylene with a mass per unit area of 10-40 g/m².

Preferably moreover provision can be made that the wound exudate absorption body thus described is fixed on at least one inside of the sleeve surrounding it, preferably by bonds.

In another embodiment provision is preferably made that the flexible foam of the sleeve is at least one material selected from the group containing thermoplastic flexible foams, such as polyurethane, polyamide or polyether foam, silicone foam as well as cellulose foam or natural sponge.

Natural sponges, for example the class of the demosponge (Demospongiae), similar to technical foams exhibit an absorption capacity for liquids. Moreover they exhibit growth-inhibiting growth-inhibiting properties vis-à-vis micro-organisms in order to protect themselves from the settling of sessile organisms. These properties can also be practical in connection with wound management in order to prevent the growth of bacteria in the wound dressing and/or in the wound. Likewise these sponges exhibit growth-inhibiting properties vis-à-vis fungi and unicellular organisms. In addition, such sponges are able to absorb liquids and therefore are ideally suited for the absorption of exudates.

The aforementioned natural sponge can be placed on the wound in thin slices which e.g. are produced by means of thermal cutting.

The flexible foam can if applicable be formed in multiple layers, wherein the individual layers can exhibit thicknesses ranging preferably between 0.5 mm and 10 mm.

The flexible foam can be open celled and closed celled in form. Moreover the flexible foam can also be designed as integral foam.

In the aforementioned embodiment provision is especially preferably made that the sleeve exhibits grooves, perforations or punches made of supporting fibers and hydroactive polymers. These facilitate the passage of liquid, in particular exudate, to the centrally located absorption body. This embodiment is advantageous in particular in the case of the use of CMC as a hydroactive polymer. CMC shrinks upon contact with liquid, which leads to an enlargement of the holes and thus additionally facilitates the passage of liquid.

Individual components of the sleeve of the sleeve can be physically joined with each other to the edges of the wound dressing, e.g. by gluing, sewing or bonding. Further physical bonding techniques are conceivable here and are recorded for the purpose of the invention. In particular thermal welding or ultrasound welding are possible welding methods.

In principle in the joining of two layers, in particular in the case of gluing, sewing or bonding an external seam is produced. Said seam can be disadvantageous in some use cases, for example whenever the wound care article is placed in a wound pocket, since here the seam protrusions constitute rubbing edges, which if applicable can result in inflammations, however at the minimum resulting in pain. For such cases provision can be made that the components of the sleeve are joined by an internal seam; such an internal seam can e.g. be produced by sewing two rectangular layers of the sleeve to three sides with each other in conventional manner, and the obtained product then “turns inside out”. In the interior thus formed a layer containing super-absorbent polymers corresponding to the above description can be. Moreover by means of the turning inside out the corners of the wound care article are rounded off (hence as it were “defused”) and the wound care article takes on an overall rounded shape which under circumstances can appear similar to e.g. a tampon. This is of advantage particularly in the case of the usage of the wound care article in wound pockets. The remaining open side can be sewed, glued or also left open. One can thus in a preferable embodiment obtain a tampon-shaped or sausage-shaped absorption body with internal seams which is well suited for being introduced into a wound pocket. If applicable said absorption body can also be provided with a strap with whose help the absorption body can be pulled out of the wound pocket after usage.

In principle in another preferable embodiment the absorption body—seen in top view on the flat side of the sleeve—can be significantly smaller than the field of the sleeve limited by the seam. In an extreme case however it can, in case it is present in the form of a mat, nearly reach the seam. With respect to the rapid increase in volume of the absorption body it is more advantageous to keep a peripheral distance between the seam of the sleeve and the lateral edges of the mat, for example ranging from 5 cm to 15 cm.

Preferably provision is further made that the sleeve is asymmetrically structured such that the region of the sleeve, which when the wound care article is placed on the body of a patient is pointing away from the body of the patient exhibits a hydrophobic, waterproof, and/or water-repellent material.

The aforementioned region of the sleeve serves in this embodiment as drench or laundry protection.

In accordance with the invention furthermore provision is made for the use of a wound care article in accordance with the invention for the treatment of chronic wounds, acutely bleeding wounds and/or traumatically induced wounds as well as the use of a wound care article in accordance with the invention for operative or post-operative care or military medical wound management and the use of a wound care article in accordance with the invention as primary or secondary wound dressing.

Further in accordance with the invention a kit is provided for the acute, emergency or military medical or chronic wound management, exhibiting a wound care article in accordance with the invention in accordance with any one of the preceding claims.

Further in accordance with the invention a method for the production of a wound care article in accordance with the invention is provided, exhibiting the following steps:
a) Provision of planar fabric bands exhibiting fibers with gel-forming properties, as well as fibers with non-gel-forming properties,
b) weaving, interweaving, stitch-bonding or knitting of these bands to a planar structure.

Preferably in the process provision is made that the used fabric bands
i) are made exclusively out of fibers with gel-forming properties or out of fibers with non-gel-forming properties, or
ii) are made both of fibers with gel-forming properties as well as also out of fibers with non-gel-forming properties

Especially preferably in the process provision is made that the method prior to Step a) includes the step
a1) chemical modification of at least a portion of the fabric band to the extent that the contained fibers exhibit at least some gel-forming properties.

In the case of the aforementioned chemical modification it is preferably a method for carboxyalkylation, especially preferably for carboxymethylation, of the cellulose or viscose, as described above. This method is suitable, as indicated above, only for dispersed cellulose fibers or for very narrow cellulose bands.

Through the chemical modification or the carboxyalkylation of the aforementioned fabric bands and the subsequent weaving of these bands to a planar structure in accordance with the invention for the first time it is possible to produce large area wound care articles exhibiting chemically modified cellulose with sizes of 10×10 cm and more.

Since carboxyalkylated cellulose (in particular carboxymethyl cellulose) is in general quite fragile, it is only possible to process carboxyalkyl cellulose filaments or yarns to planar structures afterwards with difficulties, e.g. by weaving or stitch-bonding. Thus it is only possible with difficulties to produce cellulose filaments or yarns first to carboxymethylate and then make a planar structure out of it, as suitable for usage as large area wound care article.

The above named method solves this problem by allowing a fabric band to be produced first, for example out of cellulose and then subjecting this fabric band to chemical modification in an additional step (for example carboxymethylation). In this way the aforementioned fabric band can be produced from the (still) sufficiently robust cellulose fiber (and if applicable an additional non-gel-forming fiber, such as e.g. nylon), and only subsequently is the cellulose converted to CMC. The fabric bands which now contain CMC can then be processed into planar structures as wide as one wishes.

### Variants

Preferably a provided sleeve if applicable is made at least partially also out of a hydrophobic material, for example out of polypropylene or out of a hydrophobically equipped natural material, such as cotton. The hydrophobic properties of the sleeve prevent it from adhering to the surface of the wound and assist the wound exudate in reaching the interior of the sleeve more quickly.

The sleeve can also be produced of a different synthetic material, in particular polyurethane or polyethylene film or an artificial spider silk film.

The material of the sleeve can be structured in such a way that the sleeve exhibits a rough interior surface and a smooth exterior surface. Preferably the rough interior surface of the sleeve is formed by funnel-shaped perforations which taper in the direction of the interior surface and end in a free opening edge (“Overhang”). This rough interior surface counteracts the displacements of the content of the sleeve, so that a fixation with adhesion points can be dispensed with. Accordingly the smooth exterior surface of the sleeve material can be formed by curved, material sections extending between the perforations. Such a sleeve material can, in contrast to a two-sided plane material, be referred to as “three-dimensional”, and is known e.g. from DE102006017194 of the applicant of the present application, to whose disclosure reference is made here completely.

Preferably moreover provision is made that the sleeve of a wound dressing in accordance with the invention exhibits at least in sections an adhesive coating, and to be precise preferably on the side turned away from the wound, with whose help it can be fixed in the wound region—e.g. with a bandage.

Furthermore provision can also be made that the sleeve exhibits a region protruding above the actual wound which is provided with adhesive tape for fixation.

Moreover provision a planar material section can also be provided on the side turned away from the wound of the wound dressing, said planar material section going beyond the actual sleeve and exhibiting at least in its peripheral regions an adhesive coating turned to the skin e.g. in the form of adhesive tape or adhesion surfaces (so-called “Island Dressing”).

The aforementioned planar material section can in particular exhibit semi-occlusive or semipermeable properties, i.e. it can e.g. be permeable to moisture, but not to bacteria.

The permeability for steam preferably ranges between ≧500 g to ≦16000 g m⁻² h⁻¹.

Possible adhesive materials for the above named purposes are preferably physiologically acceptable adhesive agents, such as e.g. hydrocolloid adhesive or medically harmless adhesives, such as solvent-free, biocompatible silicone adhesives or polyacrylate adhesives, which exhibit a good resistance to all common sterilization methods.

The polyurethane foam, in contrast to other synthetic materials such as polypropylene, polytetrafluorethylene (Teflon) and silicone, adheres well.

In a special embodiment provision is made that the absorption body within the sleeve is arranged asymmetrically, i.e. predominantly on one side.

Such a wound care article can preferably be used in wound pockets, where narrow spaces prevail. In the process the section of the wound dressing, in which the absorption body is located remains outside of the wound pocket. Through the capillary forces exudates which are located in such a wound pocket are effectively absorbed and here as well wound healing is promoted.

Preferably provision is furthermore made that the wound dressing exhibits a lateral notch or a wedge-shaped recess to such an extent that the borders of the notches or of the recess can be arranged overlapping.

Preferably provision is furthermore made that the wound dressing exhibits a section filling out the wound. Said section can be designed in such a way that upon contact with exudate it swells up and fills out the wound to the wound floor.

In this way the wound dressing is three-dimensionally formable, so that a concave form arises in order to adapt it e.g. for placement on a joint an extremity or a curved body part. The overlapping arranged borders of the notch or of the recess can in the process be fixed e.g. by Velcro fasteners, snap fasteners, adhesive tape or other suitable means of fixation.

In another preferable embodiment provision is made that the wound dressing is three-dimensionally formed to such an extent that it is adapted for placement on a joint, an extremity or a curved body part.

Thus the wound dressing can e.g. exhibit a concave form in order to adapt it for placement on the heel of a foot or a patient's elbow. The absorption body, which is arranged inside of the wound dressing, can in the process be designed to be removable. This embodiment is particularly advantageous when the absorption body has been moistened beforehand or moistened by leaking exudate, since a gel forms here on the basis of the superabsorbing polymers that are present, said gel having a cushioning effect and hence facilitating the pain-free support of the aforementioned body parts. Moreover the wound dressing adapts better to the anatomical circumstances through the moistening.

Preferably moreover provision is made that the wound dressing is at least partially present in rolled form. Here provision can be made that the originally planar wound dressing is rolled and if applicable in its rolled form is fixed e.g. by sewing, gluing or welding. Such a wound dressing is suitable in particular for usage as tamponade in wound pockets; in particular it exhibits a wicking function for the exudate to be absorbed.

Preferably in this connection provision is moreover made that the wound dressing exhibits in addition at least one nutritive active ingredient, at least one disinfecting or decontaminating active ingredient and/or at least one protease inhibiting active ingredient and/or active ingredient complex.

In the case of the disinfectant active ingredient and/or active ingredient complex it can for example be a composition or at least a vitamin or vitamin derivative, a metallic ion as well as a detergent. Likewise it can be a BLIS (bacteriocin like inhibitory substance), an antimicrobial peptide, an antibiotic, silver preparation or coated magnetic particle. Quaternary ammonia compounds are to be named as being especially preferable here.

In the case of the nutritive acting active ingredient and/or active ingredient complex it can be a composition containing at least the components of enteral and/or parenteral dietary agent. Likewise it can be at least one active element selected from the group containing insulin, recombinant insulin, proinsulin, an insulin-like growth factor (IGF), an insulin mimetic and/or a diabetic-specific, non-glucose-base or sucrose-base source of energy.

The nutritive acting active ingredient and/or active ingredient complex can also be a glycolysis enzyme, thus e.g. a hexokinase, which converts the glucose to glucose-6-phosphate and thus reduces the frequently increased glucose level in the case of diabetics by introduction of the glycolysis.

The protease inhibiting active ingredient and/or active ingredient complex can be at least one active element selected from the group containing protease inhibitors, super-absorbent polymers, antibodies for matrix metalloproteases (MMP, in particular for MMP 2, 7 and 9), chelators for bivalent cations (in particular for Ca²⁺), collagens, coated magnetic particles, acids, buffers, non-pathogenic acid-producing microorganisms, probiotics and/or symbiotics. In particular provision can in the process be made that in the wound dressing a carrier substance is provided which bonds bivalent cations (in particular Ca²⁺).

Additional contexts and backgrounds for the nutritive active ingredients, disinfecting or decontaminating and/or protease inhibiting active ingredients and/or active ingredient complexes are described in DE102007030931 of the applicant of the present application, to whose content reference is made here completely. In DE102007030931 further nutritive, disinfecting or decontaminating and/or protease inhibiting active substances and/or active substance complexes are described, which should likewise be considered as disclosed in this application.

Furthermore the wound dressing can contain a preparation containing phages and/or components of the same. Such a wound dressing is described in DE102007054127 of the applicant of the present invention, to whose content reference is made here completely.

Provision can moreover be made that the wound dressing contains necrolytic and/or fibrinolytic enzymes. Likewise it can contain angiogenesis or epidermogenesis promoting growth factors (in particular from the group of the VEGF and EGF). Likewise the wound dressing can contain chemical attractants for macrophages which phagocytize the redundant phages and bacterial remnants (in particular endotoxins) in phage therapy.

Furthermore provision is preferably made that at least one section of the sleeve wall of the wound dressing exhibits a reservoir for at least one nutritive, one disinfecting or decontaminating, one protease inhibiting, a hemostatic and/or wound healing active ingredient and/or active ingredient complex.

The aforementioned reservoir can e.g. consist of a pocket incorporated in the sleeve wall. Likewise the reservoir can consist of a section of the sleeve wall impregnated with the aforementioned active ingredient and/or active ingredient complex, or the active ingredient is pressed in the aforementioned section.

Substances which can increase the osmotic pressure can be added to the wound dressing. Such substances include e.g. osmodiuretics, such as Mannitol.

Mineral ion exchangers, such as zeolite, bentonite or montmorillonite can likewise be part of the wound dressing, in particular of its mat. Zeolite can among other things absorb noxious substances, such as heavy metals. Moreover they develop a hemostatic effect.

Likewise the article can also contain activated charcoal. This is preferably dispersed in a thin layer, for example in a fleece layer, arranged on the side turned away from the wound, and in particular serves the purpose of absorbing unpleasant odors from the wound region.

Furthermore the wound dressing can contain a preparation containing phages and/or components of the same. Such a wound dressing is described in DE102007054127 of the applicant of the present invention, to whose content reference is made here completely.

Furthermore the wound dressing in accordance with the invention can also be incorporated in a wound managements system for wound drainage using a vacuum. Such systems are e.g. disclosed in the publications DE202004017052, WO2006048246 and DE202004018245 of the applicant of the present invention, whose disclosure should be considered as corresponding to the present invention.

From the first named publication a device for treatment of wounds with application of a vacuum is known, exhibiting a gas tight wound dressing element which when applied to the body of the patient forms a remaining space between the wound and wound dressing element, and at least one connector point, which is in contact with the space and via which the air located in the space can be evacuated, wherein the wound dressing element is underlaid by at least one surface wound dressing absorbing the wound exudate, whose volume in the course of the absorption process increases, so that the absorbed wound exudate remains within the wound dressing and with this under the wound dressing element until the removal of the wound dressing from the body of the patient, the wound dressing is at least one layer of a textile section enriched with superabsorbers, which is enclosed by a liquid permeable sleeve, and the layer in top view has an area on its flat side which is 3% to 90% smaller than that of the sleeve so that the wound dressing can approximate a roundness in cross-section in the proximity of its total packing material capacity.

From the second named publication a multi-component dressing for wound treatment on the human or animal body with application of reduced pressure, exhibiting: a wound covering element for application to skin and mucous membrane surfaces, at least one connector point which is in contact with the wound region and by means of which the substances in the wound region can be evacuated, wherein said bandage exhibits super-absorbent polymers, wherein the absorbed wound exudate remains bound in the polymer until removal from the wound region, wherein the polymers through their binding capacity support reciprocal synergies with the subatmospheric pressures.

From the last named publication a drainage device is known for the treatment of wounds with application of a vacuum, comprising a gas-tight wound dressing element made of film-like material which when applied to the body of the patient is fixed around the wound region to the skin surface by adhesive and forms a remaining wound space between the wound and the wound dressing element, at least one drainage tube which can be inserted into the space, via which substances in the space can be evacuated, and at one wound exudate absorbent wound dressing arranged within the space which exhibits at least one layer of a textile section enriched with superabsorbers, which is surrounded by a liquid permeable sleeve, wherein the absorbed wound exudate remains within the wound dressing and with this under the wound dressing element until the removal of the wound dressing from the body of the patient, and wherein the wound dressing element exhibits a gas-tight closing treatment opening through which the wound dressing can be placed in the space and can be removed from the space.

In the process—unlike the embodiments shown in the named disclosures—moreover provision can be made that in the region of the vacuum device no absorption body exhibiting super-absorbent polymers is provided. On the contrary, what is important here is the fact that the microfibers—or a knitted fabric, spacer fabric, knitted fabrics or fleece containing microfibers—is provided as cushioning, which underlays the shell of the device and thus provides for a durable cushioning and permeability for liquids. Here in particular the dimensionally stable, non-swelling properties of the microfibers are advantageous, said microfibers exhibiting restoring forces that are sufficiently high even on application of a vacuum of conventional manner 80-250 mm Hg. With this the microfibers used in accordance with the invention constitute an outstanding replacement for the foams used here otherwise, which in particular lack the aforementioned restoring forces. A corresponding embodiment in accordance with the invention is e.g. shown in FIG. 10.

The wound dressing in accordance with the invention can moreover exhibit a form adapted to anatomical circumstances. For this purpose said wound dressing can for example be constructed in the shape of a tourniquet; which can be put over the one arm or leg or joint, or in the shape of a bandage adapted to the heel, the elbow joint or the like.

The wound dressing in accordance with the invention can moreover be constructed in such a way that it is suitable for placement around a line inserted for surgery. For this purpose the wound dressing can exhibit e.g. at least one slit, which makes it possible to place the wound dressing around a line (e.g. a drainage line or a catheter) on the body of a patient. Such a wound dressing is e.g. known from DE202006005966 of the applicant of the present invention, whose contents should be completely added to the disclosure of the present description. In the process in the distal region of the slit a web, a button, a whipped seam, a weld seam, a perforated bridge or another detachable connection (“predetermined breaking point”) can be provided which makes it possible to use the wound dressing in the usual manner or in the style of a compress with slit.

Of special importance here is the fact that the wound dressing in accordance with the invention does not swell up in the case of liquid absorption and increase in volume, since in this way it is prevented that the belted line is narrowed or blocked.

Likewise in this connection provision is preferably made that the wound dressing exhibits at least one agent which can restrict the bleeding or bleeding tendency.

The aforementioned agent can be at least one chemically and/or physiologically acting active ingredient or active ingredient complex or at least one physically acting active element. Such a wound dressing is e.g. known from DE102007030931 of the applicant of the present application.

For this purpose the wound dressing can for example
be constructed as an essentially flat material section exhibiting absorption material which is made of an absorbent fleece with superabsorbent polymers distributed within as well as at least one chemical and/or physiological active ingredient or active ingredient complex,
as or in combination with a pressure or compression bandage, in particular as part of a compression therapy in the case of leg and foot ulcers venosum
as a combination of a primary, non-absorbing or only negligibly absorbent wound dressing which exhibits at least one chemically and/or physiologically acting active ingredient or active ingredient complex and a secondary wound dressing arranged peripheral from this primary wound dressing which contains super-absorbent polymers, wherein if applicable a diffusion barrier is arranged between the two,
in the form of a first aid dressing, exhibiting a primary wound dressing with at least one chemically and/or physiologically acting active ingredient or active ingredient complex as well as a winding section arranged on the wound dressing, said winding section exhibiting at least in sections super-absorbent polymers, and/or
as a material section with a longitudinal extension exhibiting absorption material, wherein the material section exhibits elastically deformable properties, and wherein the material section exhibits super-absorbent polymers as well as if applicable at least one chemical and/or physiological active ingredient or active ingredient complex

Preferably the chemically and/or physiologically acting active ingredient or active ingredient complex includes at least one substance or a composition exhibiting hemostatic properties. These substances are known under the generic term of “hemostatics”.

Preferably the chemically and/or physiologically acting active ingredient or active ingredient complex includes at least one substance or a composition exhibiting hemostatic properties. These substances are known under the generic term of “hemostatics”.

The physically acting active element can for example be a Spanish tourniquet, a pressurized cushion, a pressure bandage or a compression bandage.

### DRAWINGS

The invention will be explained more closely in a few examples with the help of the drawings.

In FIG. 1 a planar structure 10 is shown enlarged, consisting of fabric bands 1 and 2 crossed with one another at right angles of a width of about 3 mm. The fabric band 1 is made of non-gel-forming fibers in a web technique known in and of itself, here: of Lycra fibers, on the other hand the other fabric band 2 is made of gel-forming carboxymethyl cellulose fibers. The entire mass per unit area of the planar structures 10 is about 80 g/m².

FIG. 2 shows a planar structure 20 consisting of fabric bands 3 and 4, which are likewise crossed at right angles, wherein the two fabric bands 3, 4 are each interwoven from gel-forming (gray) and non-gel-forming (black) fibers. The percentage by weight of non-gel-forming fibers is in the present case 15%. Dunova fibers are employed as non-gel-forming fibers. Dunova is a synthetic acrylic fiber which can absorb up to 35% moisture. Since the fabric bands 3 and 4 exhibit equal mechanical properties in both directions X, Y the planar structure can be termed as homogenously elastically extensible.

A sleeve wall 5.1 of a sleeve 5 shown in FIG. 3 of a pulvinated wound dressing 30 is made from fabric bands 3, 4. The sleeve wall 5.1 is intended for resting on a wound not shown in the figure, a sleeve wall 5.2 turned away from sleeve wall 5.1 consists of a hydrophobic, yet breathable polyester film. The sleeve 5 encloses an absorption body 10, which in turn consists of a synthetic wool core 7 with pulverulent superabsorber particles 8 distributed within and a liquid permeable polyester inner sleeve 6. In a further exemplary embodiment not shown in the figure the absorption body consists of an enveloped airlaid mat which is likewise enriched with superabsorber particles.

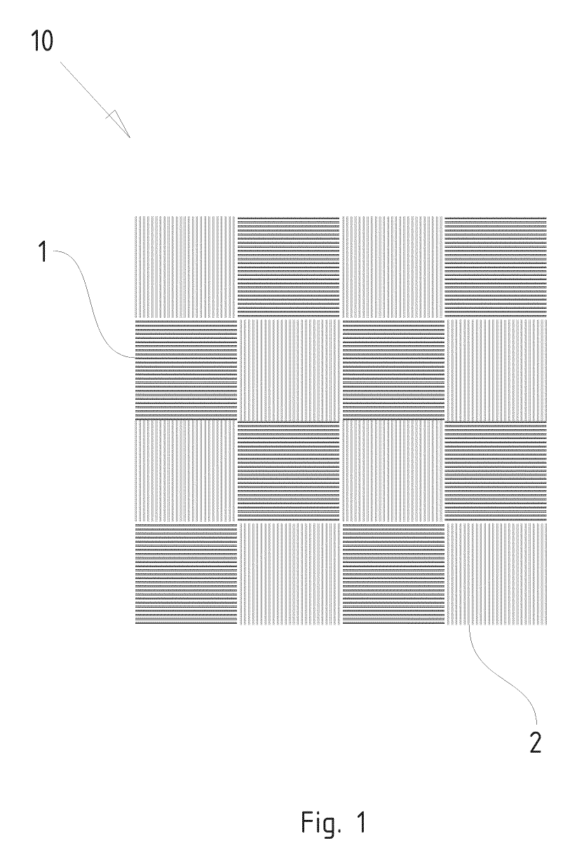

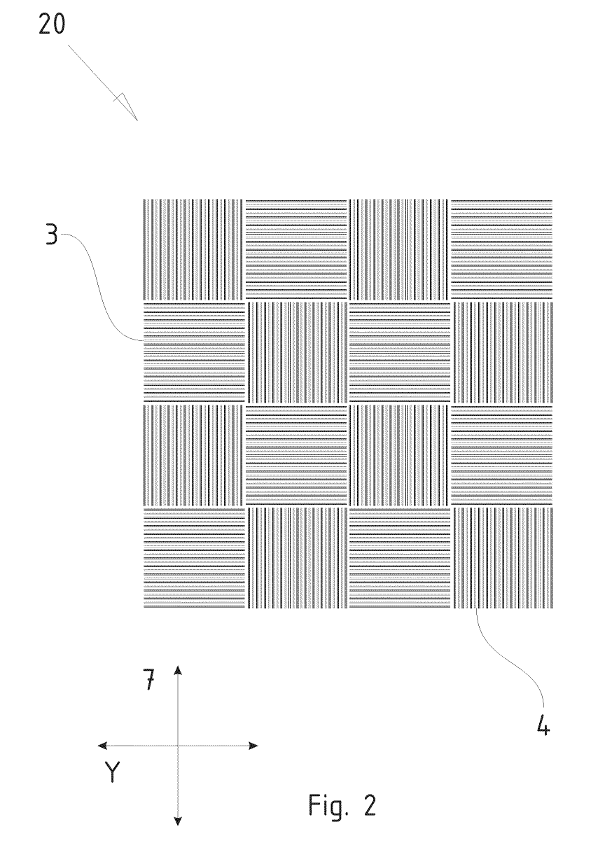

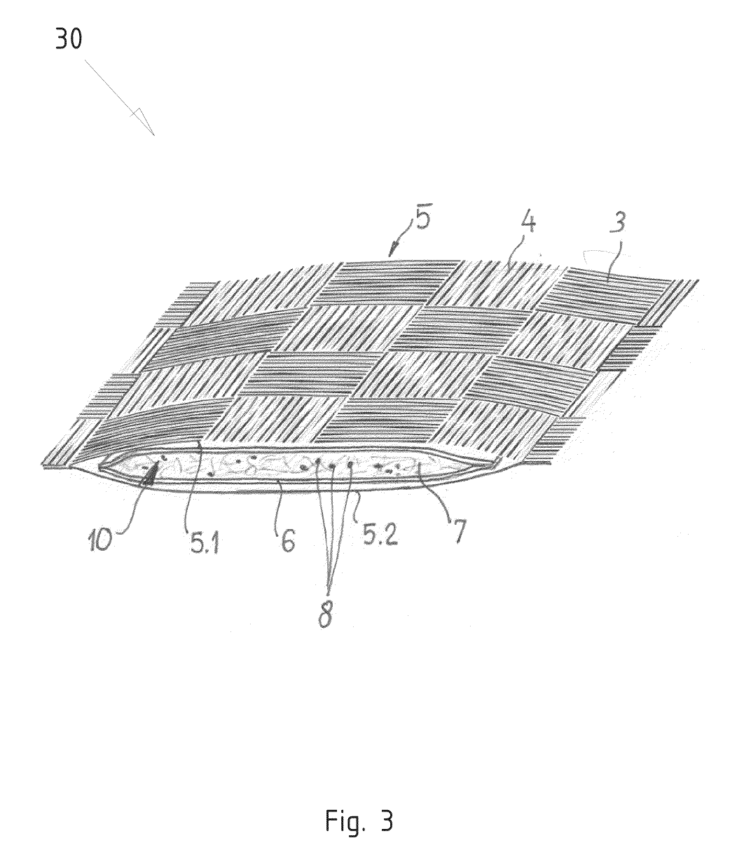

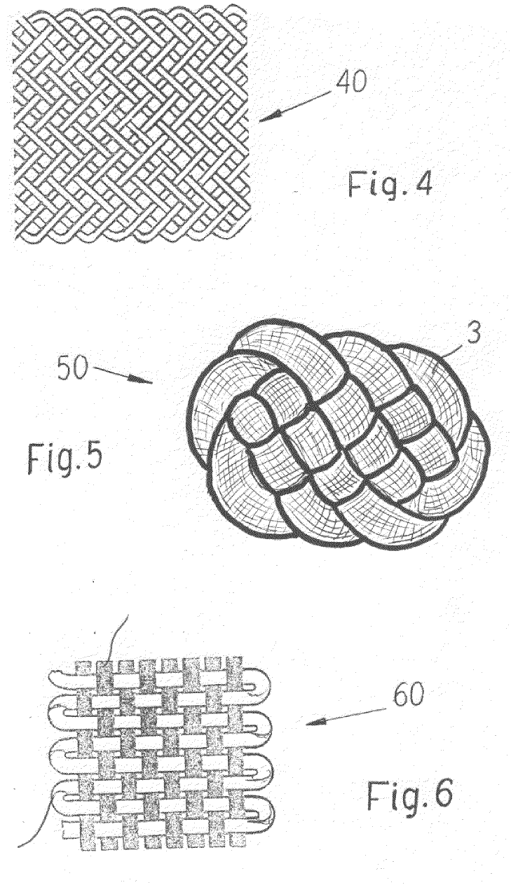

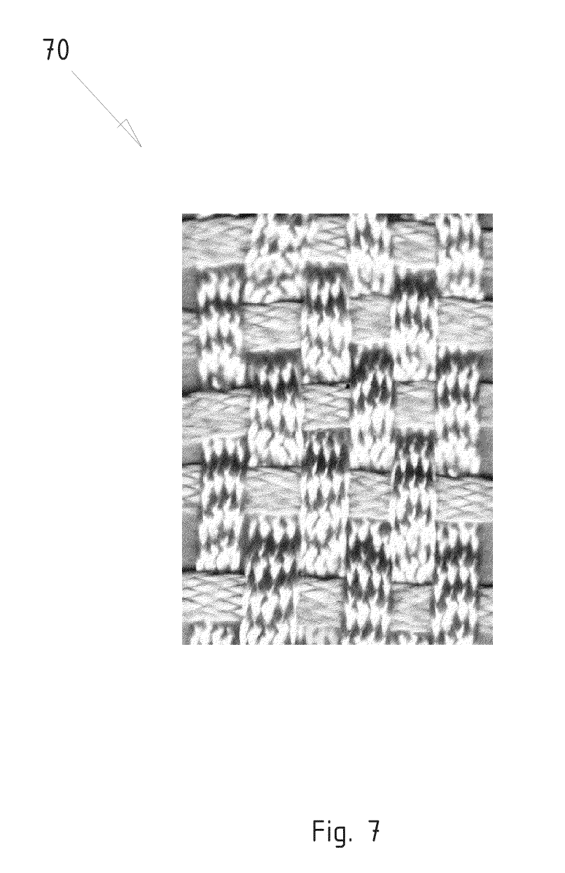

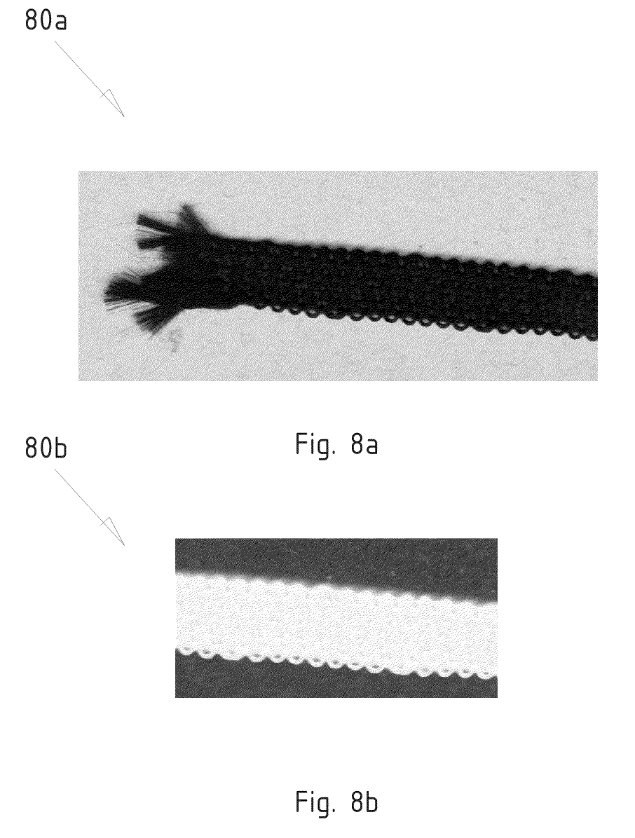

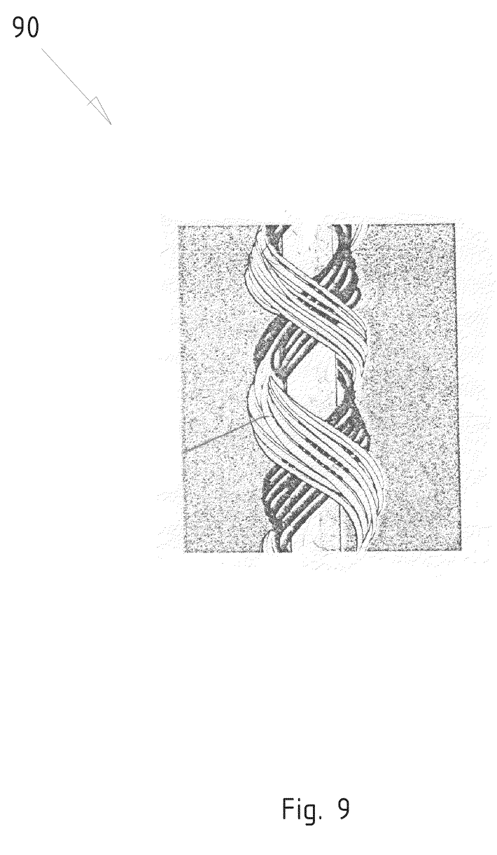

FIG. 4 shows a planar meshwork 40 consisting of diagonally crossed fabric bands 3, 4 in accordance with FIG. 2, which exhibit fibers with non-gel-forming properties, as well as fibers with gel-forming properties.

FIG. 5 shows a primary wound dressing 50, consisting of a single fabric band which is interwoven to an oval mat. The band exhibits both fibers with non-gel-forming properties as well as also fibers with gel-forming properties. The former provide the fabric band with good mechanical properties, such as tear resistance, which make it possible to easily remove the wound dressing from the wound of the patient after swelling of the gel-forming fibers while maintaining the integrity of the mat.

FIG. 6 shows a fabric 60 made from fabric bands 11, 12, wherein the fabric bands are crossed with one another at right angles. The fabric bands 12 proceed in the warp direction. The meandering-type proceeding fabric bands 11 composed of gel-forming fibers form a weft direction. The warp bands (fabric bands 12) are made of non-gel-forming Lycra fibers and extend over entire length of the band. The extensibility of the warp bands is for example 160%. The fabric bands 11 proceeding weftwise exhibit a limited elasticity or extensibility.

FIG. 7 shows a photograph of a planar structure 70 made of fabric bands in accordance with the invention, said planar structure being able to be used e.g. as a primary wound dressing or which can act as a sleeve for a wound dressing with an absorption body. The bands running in vertical direction exhibit—as fibers with non-gel-forming properties—nylon fibers, while the bands running in horizontal direction—as fibers with gel-forming properties—exhibit CMC fibers.

The latter bands can be produced from cellulose fibers which were processed unmodified into the bands, wherein the bands were subsequently carboxymethylated as a whole. As an alternative the bands can be made directly from already manufactured CMC fibers.

Here it is pointed out that individual fabric bands e.g. can also be interwoven to a tress interwoven. From such tresses, which through the partial inclination of the individual bands exhibit a three-dimensional structure, in turn planar structures can be produced. Such bands can e.g. be inserted for example in wound pockets or fistulous tracts as a wound tamponade.

Likewise the individual bands can be stitch-bonded e.g. to a planar structure (hooked, knitted).

FIG. 8a shows a photograph of a fabric band 80a made from nylon fibers which can be used within the scope of the present invention. It can be well distinguished in the left region of the image that the aforementioned band is not manufactured from yarn, but rather from monofilaments.

FIG. 8b shows a photograph of a fabric band 80b made from CMC fibers which can be used within the scope of the present invention. The band can be manufactured from cellulose fibers which have been processed unmodified into the band, wherein the bands were subsequently carboxymethylated as a whole. As an alternative the band can be made directly from already manufactured CMC fibers.

FIG. 9 shows so-called “Core Spun”—embodiment 90, in which case a core fiber is spun from a non-gel-forming material (here: Lycra) from gel-forming fibers (here: CMC monofilaments). From this “Core Spun” fiber then the fabric bands already mentioned are manufactured, which in turn are used for the manufacturing of a planar structure.

1. Wound care article, exhibiting at least one planar structure containing
a) fibers with gel-forming properties, as well as
b) fibers with non-gel-forming properties,
characterized in that the fibers are arranged in the form of planar fabric bands.

2. Wound care article in accordance with claim 1, characterized in that the wound care article exhibits several planar fabric bands, which are interwoven, interlaced, stitch-bonded or interlinked.

3. Wound care article in accordance with claim 1, characterized in that the fibers with gel-forming properties are based on chemically modified cellulose.

4. Wound care article in accordance with claim 3, characterized in that the chemically modified cellulose is carboxyalkyl cellulose or carboxyalkyl viscose.

5. Wound care article in accordance with claim 1, characterized in that the fibers with non-gel-forming properties maintain their structural integrity in spite of the influence of moisture.

6. Wound care article in accordance with claim 1, characterized in that the fibers with non-gel-forming properties exhibit at least one fiber type, selected from the group containing cellulose, viscose, linen, wool, and/or synthetic fibers.

7. Wound care article in accordance with claim 1, characterized in that the amount of fibers with gel-forming properties in aforementioned planar structure of the wound care article lies between the range of and includes 50 percent by weight and includes 100 percent by weight.

8. Wound care article in accordance with claim 1, characterized in that the aforementioned planar structure exhibiting planar fabric bands
a) forms a sleeve around an absorption body,
b) forms the shape of a dot, and/or
c) forms the shape of a wound cloth, a wound dressing, a wound compress, a wound cushion, a bandage or a stocking.

9. Wound care article in accordance with claim 1, characterized in that said wound care article exhibits super-absorbent polymers.

10. Wound care article in accordance with claim 9, characterized in that the absorption body exhibits at least one material, selected from the group containing
a) a fiber mat, in particular made of an airlaid, with incorporated superabsorbent polymers,
b) a fleece, a non-woven, an airlaid, a mat, a knitted fabric or a fabric exhibiting fibers or yarns from superabsorbent polymers,
c) an absorbent cellulose material
d) a loose packing material exhibiting super-absorbent polymers, and/or
e) a mat from flexible foam.

11. Wound care article in accordance with claim 8, characterized in that the sleeve is asymmetrically structured to such an extent that the region of the sleeve, which when the wound care article is placed on the body of a patient is pointing away from the body of the patient exhibits a hydrophobic, waterproof, and/or water-repellent material.

12. The use of a wound care article in accordance with claim 1 for the treatment of chronic wounds, acutely bleeding wounds, and/or traumatically induced wounds.

13. The use of a wound care article in accordance with claim 1 for operative or postoperative care or military medical wound management.

14. The use of a wound care article in accordance with claim 1 as a primary or secondary wound dressing.

15. Kit for the acute, emergency or military medical or chronic wound management, exhibiting a wound care article in accordance with claim 1.

16. Method for the production of a wound care article in accordance with claim 1, exhibiting the following steps:
a) Provision of planar fabric bands exhibiting fibers with gel-forming properties, as well as fibers with non-gel-forming properties,
b) weaving, interweaving, stitch-bonding or knitting of these bands to a planar structure.

17. Method in accordance with claim 16, characterized in that the used fabric bands
ii) are each made exclusively from fibers with gel-forming properties or from fibers with non-gel-forming properties, or
iii) are made both from fibers with gel-forming properties as well as also from fibers with non-gel-forming properties

18. Method in accordance with claim 16, exhibiting prior to Step a) the Step a1) chemical modification of at least a portion of the fabric band to the extent that the fibers contained within exhibit at least some gel-forming properties.

19. Wound care article in accordance with claim 2, characterized in that the fibers with gel-forming properties are based on chemically modified cellulose.

20. Wound care article in accordance with claim 19, characterized in that the chemically modified cellulose is carboxyalkyl cellulose or carboxyalkyl viscose.
